(19) United States
(12) Patent Application Publication
(10) Pub. No.: US 2024/0287613 A1
(43) Pub. Date: Aug. 29, 2024

(54) CANCER EARLY DETECTION METHOD USING LIQUID BIOPSY

(71) Applicant: Pharus Taiwan, Inc., Hsinchu County (TW)

(72) Inventors: Shih-Ting Kang, Hsinchu County (TW); Yu-Chuan Chang, Hsinchu County (TW); Tsung-Ting Hsieh, Hsinchu County (TW); Ko-Han Lee, Hsinchu County (TW)

(73) Assignee: Pharus Taiwan, Inc., Hsinchu County (TW)

(21) Appl. No.: 18/305,396

(22) Filed: Apr. 24, 2023

(30) Foreign Application Priority Data
Feb. 24, 2023 (TW) 112106882

(51) Int. Cl. C12Q 1/6886; G16H 50/20

(52) U.S. Cl. CPC C12Q 1/6886; G16H 50/20; C12Q 2600/112; C12Q 2600/158; C12Q 2600/178

(57) ABSTRACT

The invention provides a cancer early detection method using liquid biopsy, the cancer early detection method includes the following steps. MicroRNA expression profile database of cancer patient populations and healthy populations are established. Afterwards, an analysis model for cancer early detection is established through the following steps: a data quality control, a technical replicate merging, a calculation of normalization factors and data normalization, a biomarker feature selection, and a hyperparameter tuning. The analysis model for cancer early detection includes normalization factors, a set of biomarkers, model weights and model hyperparameters. A microRNA expression profile in a liquid biopsy sample of a subject is analyzed by the analysis model for cancer early detection to be used as a basis for an early detection of cancer.

### CROSS-REFERENCE TO RELATED APPLICATION

This application claims the priority benefit of Taiwan application serial no. 112106882, filed on Feb. 24, 2023. The entirety of the above-mentioned patent application is hereby incorporated by reference herein and made a part of this specification.

### BACKGROUND

### Technical Field

The disclosure relates to a cancer early detection method, and particularly to a cancer early detection method using a liquid biopsy.

### Description of Related Art

MicroRNA is a non-coding RNA with a length of about 18 to 25 nucleotides. MicroRNA is highly preserved during the evolution process, and plays a very important role in the regulation of cells. In 1993, miRNA was first discovered in C. elegans. One after another, more and more miRNAs have been discovered in humans and other species. At present, there are about 2,500 known miRNAs in human cells. These miRNAs have been proven to regulate more than 50% messenger RNAs' (mRNAs′) expression. Moreover, abnormal miRNA expression has been proved to be closely related to the formation of many diseases, such as cancer diseases, chronic diseases, and autoimmune diseases.

In the past few years, miRNA has been widely praised and is regarded as a new target for molecular detection. At present, miRNA has also been confirmed to be secreted from cells into the blood and form protein-RNA complexes, ensuring that it will not be degraded by ribonuclease (RNase). Such features have also become very valuable, making free miRNAs in the blood relatively easy to obtain, and may be used as a basis for initial diagnosis of diseases by detecting cell-free miRNA expression level. For example, different types of cancers have been confirmed to have their own unique expression profiles of free miRNA, or miRNA signatures, which may be used as the basis for the initial diagnosis of cancer.

For a long time, non-invasive disease detection methods that are convenient and have high diagnostic rate have been a constantly pursued goal of the medical community. Taking cancer as an example, in order to find potentially undetected and early-stage asymptomatic cancers early, cancer screening may be performed to achieve this goal. Cancer screening refers to the process of using examinations, tests, or other methods to identify the possible presence or absence of cancer.

Currently, patients may be tested for cancer via many symptoms or test results. However, the most certain way to diagnose malignant tumors is to confirm the presence of cancer cells by pathologists in biopsy or surgically obtained tissues, which is an invasive detection method.

In addition, tumor marker detection refers to the determination of cancer by detecting changes in special proteins associated with malignant tumor cells. However, the sensitivity and specificity of tumor marker detection is not good, and tumor is often detected when the tumor has already developed to a considerable size or has metastasized to other organs.

Based on the above, the development of a non-invasive cancer early detection method for early evaluation, determining whether the subject has cancer, and early diagnosis and treatment are important topics for current research.

### SUMMARY

The invention provides a cancer early detection method using a liquid biopsy detecting early-stage cancer by analyzing the miRNA expression profile of a liquid biopsy sample of a subject.

A cancer early detection method of the invention includes the following steps. MicroRNA expression profile database of cancer patient populations and healthy populations are established. Afterwards, an analysis model for cancer early detection is established through the following steps: a data quality control, a technical replicate merging, a calculation of normalization factors and data normalization, a biomarker feature selection, and a hyperparameter tuning. The analysis model for cancer early detection includes normalization factors, a set of biomarkers, model weights and model hyperparameters. A microRNA expression profile in a liquid biopsy sample of a subject is analyzed by the analysis model for cancer early detection to be used as a basis for an early detection of cancer.

In an embodiment of the invention, the miRNA expression profile is determined by qPCR, sequencing, microarray, or RNA-DNA hybrid capture technology.

In an embodiment of the invention, the miRNA expression profile is determined by performing qPCR on a cDNA synthesized from a miRNA in the liquid biopsy sample.

In an embodiment of the invention, the miRNA expression profile comprises an expression level of a plurality of miRNAs.

In an embodiment of the invention, a type of the early detection of cancer comprises lung cancer.

In an embodiment of the invention, the liquid biopsy sample comprises plasma, serum, or urine, and exosomes further purified from the liquid biopsy sample.

In an embodiment of the invention, the analysis model for cancer early detection is established based on a classification algorithm, and the classification algorithm includes Logistic Regression or Random Forest.

In an embodiment of the invention, the data quality control comprises checking a ratio (a missing value ratio) of any miRNA in the miRNA expression profile database which does not have an expression level (a missing value) in all training dataset samples, and the miRNA of which the missing value ratio is 0 can be used as a normalization factor, if the normalization factor of a test sample contains the missing value, it is judged as failing the data quality control and removed from a testing dataset.

In an embodiment of the invention, when there are technical replicate data of a same sample in a training dataset or a testing dataset, the technical replicate merging is performed.

In an embodiment of the invention, the calculation of the normalization factor and the data normalization is to reduce a deviation between samples or batches by adjusting a data distribution or a normalization factor of a sample to be consistent.

In an embodiment of the invention, the biomarker feature selection is to remove noises caused by non-correlated features according to a weight or an importance of features.

In an embodiment of the invention, the hyperparameter tuning is used to find out a hyperparameter setting suitable for a data set.

Based on the above, the invention provides a non-invasive cancer early detection method for early evaluation, in which the miRNA expression profile of a liquid biopsy sample of a subject is analyzed via the analysis model for cancer early detection. Therefore, early cancer screening and evaluation may be performed in time and efficiently, and the convenience and detection rate of conventional cancer screening methods may be improved.

To make the aforementioned more comprehensible, several embodiments accompanied with drawings are described in detail as follows.

### BRIEF DESCRIPTION OF THE DRAWINGS

The accompanying drawings are included to provide a further understanding of the disclosure, and are incorporated in and constitute a part of this specification. The drawings illustrate exemplary embodiments of the disclosure and, together with the description, serve to explain the principles of the disclosure.

FIG. 1 is a receiver operating characteristic curve of K-fold cross-validation.

FIG. 2 is a receiver operating characteristic curve of an isolated test data (testing dataset).

### DESCRIPTION OF THE EMBODIMENTS

In the following, the embodiments of the invention are described in detail. However, the embodiments are exemplary, and the invention is not limited thereto.

The invention provides a cancer early detection method. In the following, the terms used in the specification are defined first.

‘cDNA’ (complementary DNA) refers to complementary DNA generated by performing reverse transcription on an RNA template using reverse transcriptase.

‘qPCR’ or ‘real-time quantitative PCR’ (real-time quantitative polymerase chain reaction) refers to an experimental method of using PCR to amplify and quantify target DNA at the same time. Quantification is performed using a plurality of measuring chemical substances (including, for example, fluorescent dye of SYBR® green or fluorescent report oligonucleotide probe of Taqman probe), and real-time quantification is performed with the amplified DNA accumulated in the reaction after every amplification cycle.

The term ‘expression’ refers to the transcription and/or accumulation of RNA molecules in a biological sample, such as a liquid biopsy sample. In this context, the term ‘miRNA expression’ refers to one or a plurality of miRNAs in a biological sample, and the miRNA expression may be detected by using a suitable method known in the art.

The term ‘microribonucleic acid’ (‘microRNA’ or ‘miRNA’) refers to a type of non-coding RNA with a length of about 18 to 25 nucleotides derived from an endogenous gene. miRNA acts as a post-transcriptional regulator of gene expression via base pairing with the 3′-untranslated region (UTR) of the target mRNA thereof for mRNA degradation or translation inhibition.

The terms ‘nucleic acid’, ‘nucleotide’, and ‘polynucleotide’ are used interchangeably and refer to a polymer of DNA or RNA in single-stranded or double-stranded form. Unless stated otherwise, these terms encompass polynucleotides containing known analogs of natural nucleotides that have binding properties similar to a reference nucleic acid and are metabolized in a manner similar to naturally occurring nucleotides.

The term ‘primer’ refers to an oligonucleotide used to initiate the synthesis of complementary nucleic acid strands when under conditions that induce the synthesis of a primer extension product, for example, when the oligonucleotide is placed in the presence of a nucleotide and a polymerization inducer (such as DNA or ribonucleic acid polymerase) and at a suitable temperature, pH, metal ion concentration, and salt concentration.

The term ‘probe’ refers to a structure including a polynucleotide and contains a nucleic acid sequence complementary to a nucleic acid sequence present in a target nucleic acid analyte (for example, a nucleic acid amplification product). The polynucleotide region of the probe may be composed of DNA and/or RNA and/or synthetic nucleotide analogs. The length of the probe is usually compatible with all or part of the target sequence used for the specific detection of the target nucleic acid.

The term ‘targeting’ refers to the selection of a suitable nucleotide sequence hybridizing with a nucleic acid sequence of interest.

A cancer early detection method of the invention includes the following steps. MicroRNA expression profile database of cancer patient populations and healthy populations are established. Afterwards, an analysis model for cancer early detection is established through the following steps: a data quality control, a technical replicate merging, a calculation of normalization factors and data normalization, a biomarker feature selection, and a hyperparameter tuning. The analysis model for cancer early detection includes normalization factors, a set of biomarkers, model weights and model hyperparameters. A microRNA expression profile in a liquid biopsy sample of a subject is analyzed by the analysis model for cancer early detection to be used as a basis for an early detection of cancer. In more detail, the liquid biopsy sample includes plasma, serum, or urine, and exosomes further purified from these samples. However, the invention is not limited thereto. Hereinafter, each of the above steps will be described in detail.

In the embodiment, the microRNA expression profile database is to detect expression profiles of 167 microRNAs highly related to cancer. The employed 167 microRNAs highly related to cancer are shown in Table 1 below.

TABLE 1

| No. | miRNA |
|---|---|
| 1 | hsa-let-7d-3p |
| 2 | hsa-miR-101-3p |
| 3 | hsa-miR-122-5p |
| 4 | hsa-miR-1254 |
| 5 | hsa-miR-125b-5p |
| 6 | hsa-miR-126-3p |
| 7 | hsa-miR-1290 |
| 8 | hsa-miR-129-5p |
| 9 | hsa-miR-140-5p |
| 10 | hsa-miR-142-3p |
| 11 | hsa-miR-143-3p |
| 12 | hsa-miR-144-3p |
| 13 | hsa-miR-145-5p |
| 14 | hsa-miR-146a-5p |
| 15 | hsa-miR-150-5p |
| 16 | hsa-miR-151a-3p |
| 17 | hsa-miR-152-3p |
| 18 | hsa-miR-155-5p |
| 19 | hsa-miR-15a-5p |
| 20 | hsa-miR-15b-5p |
| 21 | hsa-miR-17-3p |
| 22 | hsa-miR-181a-5p |
| 23 | hsa-miR-181b-5p |
| 24 | hsa-miR-182-5p |
| 25 | hsa-miR-183-5p |
| 26 | hsa-miR-193a-3p |
| 27 | hsa-miR-1972 |
| 28 | hsa-miR-197-3p |
| 29 | hsa-miR-198 |
| 30 | hsa-miR-199a-5p |
| 31 | hsa-miR-19a-3p |
| 32 | hsa-miR-19b-3p |
| 33 | hsa-miR-202-3p |
| 34 | hsa-miR-203a-3p |
| 35 | hsa-miR-205-5p |
| 36 | hsa-miR-206 |
| 37 | hsa-miR-20a-5p |
| 38 | hsa-miR-2114-3p |
| 39 | hsa-miR-940 |
| 40 | hsa-miR-214-3p |
| 41 | hsa-miR-215-5p |
| 42 | hsa-miR-21-5p |
| 43 | hsa-miR-216a-5p |
| 44 | hsa-miR-222-3p |
| 45 | hsa-miR-22-3p |
| 46 | hsa-miR-224-5p |
| 47 | hsa-miR-24-3p |
| 48 | hsa-miR-26a-5p |
| 49 | hsa-miR-28-3p |
| 50 | hsa-miR-299-5p |
| 51 | hsa-miR-29a-5p |
| 52 | hsa-miR-30b-5p |
| 53 | hsa-miR-31-5p |
| 54 | hsa-miR-326 |
| 55 | hsa-miR-335-5p |
| 56 | hsa-miR-338-5p |
| 57 | hsa-miR-34a-5p |
| 58 | hsa-miR-361-5p |
| 59 | hsa-miR-372-3p |
| 60 | hsa-miR-373-3p |
| 61 | hsa-miR-375 |
| 62 | hsa-miR-378a-5p |
| 63 | hsa-miR-382-5p |
| 64 | hsa-miR-409-3p |
| 65 | hsa-miR-425-3p |
| 66 | hsa-miR-452-3p |
| 67 | hsa-miR-483-5p |
| 68 | hsa-miR-484 |
| 69 | hsa-miR-499a-5p |
| 70 | hsa-miR-500a-5p |
| 71 | hsa-miR-574-3p |
| 72 | hsa-miR-574-5p |
| 73 | hsa-miR-579-3p |
| 74 | hsa-miR-589-5p |
| 75 | hsa-miR-593-5p |
| 76 | hsa-miR-596 |
| 77 | hsa-miR-601 |
| 78 | hsa-miR-1225-3p |
| 79 | hsa-let-7b-5p |
| 80 | hsa-let-7c-5p |
| 81 | hsa-let-7d-5p |
| 82 | hsa-let-7f-5p |
| 83 | hsa-let-7g-5p |
| 84 | hsa-miR-100-5p |
| 85 | hsa-miR-106a-5p |
| 86 | hsa-miR-106b-5p |
| 87 | hsa-miR-10a-5p |
| 88 | hsa-miR-10b-5p |
| 89 | hsa-miR-1248 |
| 90 | hsa-miR-125a-5p |
| 91 | hsa-miR-127-3p |
| 92 | hsa-miR-128-3p |
| 93 | hsa-miR-130a-3p |
| 94 | hsa-miR-130b-3p |
| 95 | hsa-miR-133a-3p |
| 96 | hsa-miR-133b |
| 97 | hsa-miR-134-5p |
| 98 | hsa-miR-135a-5p |
| 99 | hsa-miR-135b-5p |
| 100 | hsa-miR-1-3p |
| 101 | hsa-miR-141-3p |
| 102 | hsa-miR-146b-5p |
| 103 | hsa-miR-17-5p |
| 104 | hsa-miR-18a-5p |
| 105 | hsa-miR-18b-5p |
| 106 | hsa-miR-192-5p |
| 107 | hsa-miR-193b-3p |
| 108 | hsa-miR-195-5p |
| 109 | hsa-miR-196a-5p |
| 110 | hsa-miR-196b-5p |
| 111 | hsa-miR-1973 |
| 112 | hsa-miR-199a-3p |
| 113 | hsa-miR-200a-3p |
| 114 | hsa-miR-200b-3p |
| 115 | hsa-miR-200c-3p |
| 116 | hsa-miR-208b-3p |
| 117 | hsa-miR-223-5p |
| 118 | hsa-miR-20b-5p |
| 119 | hsa-miR-210-3p |
| 120 | hsa-miR-217 |
| 121 | hsa-miR-221-3p |
| 122 | hsa-miR-223-3p |
| 123 | hsa-miR-27a-3p |
| 124 | hsa-miR-29a-3p |
| 125 | hsa-miR-29b-3p |
| 126 | hsa-miR-29c-3p |
| 127 | hsa-miR-302d-3p |
| 128 | hsa-miR-30a-5p |
| 129 | hsa-miR-30c-5p |
| 130 | hsa-miR-30d-5p |
| 131 | hsa-miR-30e-5p |
| 132 | hsa-miR-31-3p |
| 133 | hsa-miR-320a |
| 134 | hsa-miR-330-5p |
| 135 | hsa-miR-376c-3p |
| 136 | hsa-miR-411-5p |
| 137 | hsa-miR-429 |
| 138 | hsa-miR-4306 |
| 139 | hsa-miR-450a-5p |
| 140 | hsa-miR-486-5p |
| 141 | hsa-miR-518a-5p |
| 142 | hsa-miR-520b |
| 143 | hsa-miR-539-5p |
| 144 | hsa-miR-625-5p |
| 145 | hsa-miR-652-3p |
| 146 | hsa-miR-660-5p |
| 147 | hsa-miR-663a |
| 148 | hsa-miR-718 |
| 149 | hsa-miR-7-5p |
| 150 | hsa-miR-760 |
| 151 | hsa-miR-885-5p |
| 152 | hsa-miR-92a-3p |
| 153 | hsa-miR-95-3p |
| 154 | hsa-miR-9-5p |
| 155 | hsa-miR-99b-5p |
| 156 | hsa-miR-4772-3p |
| 157 | hsa-miR-451a |
| 158 | hsa-miR-23a-3p |
| 159 | hsa-let-7a-5p |
| 160 | hsa-miR-103a-3p |
| 161 | hsa-miR-16-5p |
| 162 | hsa-miR-191-5p |
| 163 | hsa-miR-423-3p |
| 164 | hsa-miR-423-5p |
| 165 | hsa-miR-93-5p |
| 166 | hsa-miR-425-5p |
| 167 | hsa-miR-1228-5p |

In the present embodiment, the method of detecting miRNA in plasma includes the following steps:

### 1. Collection of Blood Sample

A blood sampler's skin was wiped with alcohol on the blood collection site, and a tourniquet was tied 5 cm to 15 cm above the blood collection site with a slip knot. 10 ml of whole blood was drawn into a K₂EDTA BD Vacutainer tube using a 19 G to 22 G needle. When blood flows into the blood collection tube, the tourniquet should be released immediately. After the blood draw was completed, the blood collection tube was immediately turned upside down and mixed lightly 5 to 8 times to ensure that the anticoagulant was fully functional. The blood collection tube was stored at room temperature, and the plasma separation step was completed within one hour after blood collection.

### 2. Plasma Separation Method

The blood collection tube was placed on a swinging-bucket rotor and centrifuged at 1200×g for 10 minutes at room temperature. After centrifugation was completed, the supernatant was taken out to a new 15 ml centrifuge tube. The 15 ml centrifuge tube was pipetted 5 times to ensure even mixing, and then was evenly divided into 1.5 ml DNase/RNase-free Eppendorf, and centrifuged at 12,000×g for 10 minutes at room temperature. After centrifugation was completed, the supernatant was taken out and transferred to a new 15 ml centrifuge tube to avoid taking the white sediment at the bottom of the 1.5 ml Eppendorf. The supernatant was pipetted 5 times to ensure even mixing, dispensed into 1.5 ml DNA LoBind Tubes (Eppendorf, 22431021), and stored immediately in a refrigerator at −80° C.

3. miRNA Extraction Method

The plasma sample was taken out from the refrigerator at −80° C., thawed on ice, and subjected to an experiment in accordance with the operation manual provided by Qiagen miRNeasy Serum/Plasma Kit after thawing, and then reconstituted with 30 μl nuclease-free water.

4. cDNA Synthesis

A suitable amount of miRNA was taken and a reverse transcription reaction was performed using OncoSweep microRNA Universal RT kit to synthesize cDNA.

5. qPCR Experiment

A suitable amount of cDNA was taken to perform qPCR experiment in accordance with the operation manual provided by OncoSweep PanelChip®.

In the present embodiment, the identification of early cancer screening may include cancer or healthy subjects, for example, may include lung cancer or healthy subjects. However, the invention is not limited thereto, and may also include other cancers, tumor risks, or risk factors for cancer. More specifically, for the miRNA database for different cancer populations and healthy populations and the analysis model for cancer early detection established thereby, according to the miRNA expression profile of the subject, the diagnosis prediction may be divided into lung cancer or healthy subjects, but the invention is not limited thereto. More specifically, the expression profile of miRNA may be determined by, for example, qPCR, sequencing, microarray, or RNA-DNA hybrid capture technology, preferably, for example, by performing qPCR on cDNA synthesized from miRNA in a liquid biopsy sample. The miRNA expression profile may include an expression level of a plurality of miRNAs.

In the present embodiment, the analysis model for cancer early detection is established based on a classification algorithm, which may include Logistic Regression or Random Forest. In more detail, the following steps are performed to construct the analysis model for cancer early detection: a data quality control, a technical replicate merging, a calculation of normalization factors and data normalization, a biomarker feature selection, and a hyperparameter tuning, and the analysis model for cancer early detection includes normalization factors, a set of biomarkers, model weights and model hyperparameters.

In the present embodiment, K-fold cross validation can also be used to verify the analysis model for cancer early detection, and to verify the isolated test data collected at different times or additionally. The verification indicators may include accuracy, sensitivity, specificity, positive predictive value (PPV), negative predictive value (NPV), and area under the curve of receiver operating characteristic curve (AUC ROC).

### Data Quality Control and Compensation for Missing Values

The purpose of quality control is to confirm whether the quality of the data is sufficient for the model. In the present embodiment, a ratio (a missing value ratio) of any miRNA in the database which does not have an expression level (a missing value) in all training dataset samples is checked, and the miRNA of which the missing value ratio is 0 can be eligible as a candidate of normalization factor. In the testing dataset, if the normalization factor of a test sample contains the missing value, it is judged as failing the data quality control and removed from the testing dataset. If the microRNAs of other non-normalization factors are missing values, the missing values will be treated as no signal, and a maximum quantification cycle (Cq) of 40 will be given as the missing value compensation. The missing value ratio of 0% and the quantification cycle of 40 are just examples, and the present invention is not limited thereto.

### Technical Replicate Merging

If there are technical replicate data of the same sample in the training dataset or testing dataset, the technical replicate merging will be performed in the embodiment. In more detail, the median of each microRNA quantification cycle will be taken as the merged quantification cycle. The merging method for medians above is just an example, but the present invention is not limited thereto, and technical replicate merging can also be performed by methods such as mean.

### Calculation of Normalization Factors and Data Normalization

The purpose of data normalization is to reduce the bias between samples or batches by adjusting the data distribution or normalization factors of samples to be consistent. This embodiment uses the normalization factor algorithm NormFinder published by Andersen C. L. et al (2004) to calculate and find the normalization factor. The normalization factor refers to microRNAs with stable quantification cycle and small variance between samples and between groups and groups. If the quantification cycle of the plurality of microRNAs are stable and have little variation, the average value of the quantification cycle is the value of the normalization factor. Finally, the normalized training set and testing set data are calculated by the difference between the quantification cycle and the value of the normalization factor of all microRNAs in the training set and the testing set. The above-mentioned normalization factor algorithm is only a special example of implementation, but the present invention is not limited thereto, and can also be carried out by common methods such as Min-Max Normalization or Quantile Normalization for data normalization.

### Biomarker Feature Selection

In the field of machine learning, feature selection refers to removing the bad feature from the feature set according to their weight or importance, and removing the noise caused by non-correlated features to further improve the performance of the prediction model. The feature in this embodiment is the quantification cycle of the microRNA, and the feature selection method used is the Recursive Feature Elimination (RFE) method proposed by Guyon et al (2002). The algorithm first uses all N features to train the model, removes the feature with the smallest weight after the weights (importances) of N features are obtained, and then use N−1 features to train the model, and so on to recursively reduce the feature set to train the model until up to a preset minimum number of features. The model with the best performance trained by different numbers of feature sets is the best model, and the features used are the best feature set. In this embodiment, the 167 microRNAs in the training dataset are used for feature selection, and the model has the optimal area under the curve of receiver operating characteristic curve when 9 features are used. The best feature set selected from the training dataset will be used for cancer screening determination of the testing dataset samples. The above-mentioned Recursive Feature Elimination method is only an embodiment of feature selection, but the present invention is not limited thereto. Other ways of screening features with reference to the weight or importance of features can be used as a feature selection method.

### Hyperparameter Tuning

Hyperparameters refer to parameters that can adjust machine learning algorithms. The intuitive meanings are usually the complexity of the prediction model, the training speed of the model, and the sensitivity to data. The process of finding the most suitable hyperparameter setting for a data set is called hyperparameter tuning, and the method used in this embodiment is Grid Search algorithm. The concept is to expand the combinations of all hyperparameters, and use all the hyperparameter combinations to train models respectively, wherein the parameters used by the model with the best performance are the optimal hyperparameter settings. Taking the Random Forest algorithm used in this embodiment as an example, the optimal hyperparameter setting of the model with 9 features obtained by the Grid Search algorithm is: [criterion=‘entropy’, max_depth=5, min_samples_leaf=3, n_estimators=200]. Under this parameter, the maximum area under the curve of receiver operating characteristic curve can be obtained. The above-mentioned Grid Search algorithm is only an example of hyperparameter tuning, but the present invention is not limited thereto, and other methods such as Random Search can also be used as a hyperparameter tuning method.

### Analysis Model for Cancer Early Detection

The training dataset establishes the analysis model for cancer early detection through the above-mentioned analysis steps, and the content of the analysis model for cancer early detection includes: 1. normalization factors; 2. a set of biomarkers (for example, there may be 9 for lung cancer embodiments, but the present invention is not limited thereto); 3. model weights; 4. model hyperparameters. Taking the early screening of lung cancer as an example, the testing dataset samples will use items 1 to 3 when determining whether there is lung cancer. The first item is used for data normalization, and the second and third items are used to determine the probability of the sample suffering from lung cancer. Item 4 finds the best hyperparameters in the training dataset. After training the model with this set of hyperparameters, even if the analysis model is in a fixed manner, subsequent determinations are not directly related to hyperparameters. In this embodiment, the lung cancer embodiment can generate 3 normalization factors, and the biomarker feature can select 9 biomarkers. For example, 9 biomarkers most correlated with lung cancer can be found through the analysis model for cancer early detection, in other words, 9 biomarkers for determining lung cancer can be found, but the present invention is not limited thereto.

### Performance Evaluation Method of Analysis Model for Cancer Early Detection

The performance of the analysis model can be evaluated by K-fold cross-validation of the training dataset, and additional isolated test data collected at different times (ie, testing dataset). The K-fold cross-validation method first randomly divides the training dataset into K equal parts, retains one of the equal parts as the testing data, and makes the other K−1 equal parts as the training data; then reserves the other part in the next fold as the testing data, and the other K−1 aliquots are the training data. This process is repeated K times, and each aliquot is used as the training data once, so it is called K-fold cross-validation. However, K-fold cross-validation is usually an adaptation when there is a lack of isolated testing data and the performance of the model must be evaluated. A more objective and complete evaluation will use training data to train the model and use additional isolated test data collected at different times or locations to verify the model. Therefore, this embodiment covers two verification outcomes.

The indicators used to evaluate the analysis model in this embodiment include: accuracy (formula 1), sensitivity (formula 2), specificity (formula 3), positive predictive value (formula 4), negative predictive value (formula 5) and area under the curve of receiver operating characteristic curve. The receiver operating characteristic curve refers to the curve composed of the true positive rate and the false positive rate under different classification thresholds, wherein the X-axis is the false positive rate and the Y-axis is the true positive rate. The true positive rate is equal to sensitivity and the false positive rate is equal to (1−specificity). The closer the receiver operating characteristic curve is to the upper left corner, the better the performance, which can intuitively and visually identify the pros and cons of the model. The area under the curve of receiver operating characteristic curve, as the name implies, is the area under the curve of above operating characteristic curve. Its geometric meaning is that the larger the area under the curve, the better the performance of the model. If the area under the curve is 1, it represents the model perfect classification; if the area under the curve is 0.5, it means the model is randomly classified.

$$Accuracy = \frac{True Positive + True Negative}{Condition Positive + Condition Negative} \qquad formula 1$$

$$Sensitivity = \frac{True Positive}{Condition Positive} = \frac{True Positive}{True Positive + False Negative} \qquad formula 2$$

$$Specificity = \frac{True Negative}{Condition Negative} = \frac{True Negative}{True Negative + False Positive} \qquad formula 3$$

$$Positive Predictive Value = \frac{True Positive}{True Positive + False Positive} \qquad formula 4$$

$$Negative Prediction Value = \frac{True Negative}{True Negative + False Negative} \qquad formula 5$$

In the following, the performance evaluation of the analysis model for cancer early detection by the K-fold cross-validation of the training dataset in the present invention is presented using the data.

### Example 1: Evaluation for Lung Cancer Early Detection

The training dataset uses plasma samples from 85 subjects known to be early-stage lung cancer (stage 1-2 judged by physicians) and 99 subjects known to be healthy (not yet diagnosed with any major disease). MicroRNA expression profile analysis is performed, and the 184 cases of data is used as a training set to train the model. Healthy people refer to subjects who have not been diagnosed with any major diseases by physicians, and those with early-stage lung cancer have not received treatment. The K-fold cross-validation results of the analysis model for lung cancer early detection are obtained and shown in Table 2.

TABLE 2

| | | | | | | The area |
|---|---|---|---|---|---|---|
| | | | | | | under the |
| | | | | | | curve of |
| | | | | | | receiver |
| | | | | Positive | Negative | operating |
| | | | | predictive | predictive | characteristic |
| Training set | Accuracy | Sensitivity | Specificity | value | value | curve |
| 10-fold | 0.8750 | 0.8353 | 0.9091 | 0.8875 | 0.8654 | 0.9394 |
| cross- | | | | | | |
| validation | | | | | | |

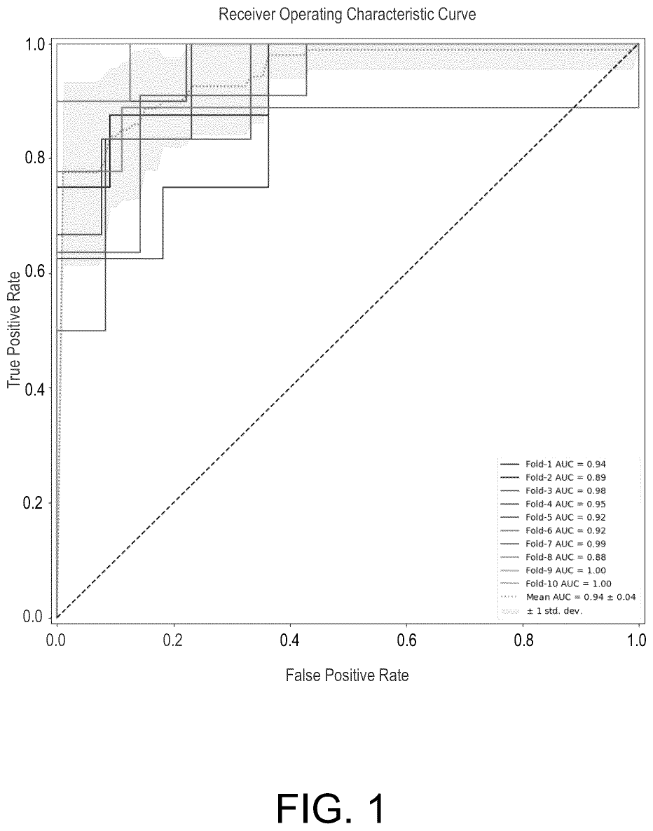

FIG. 1 is a receiver operating characteristic curve of K-fold cross-validation (training set data validation). The curves marked with different gray levels in FIG. 1 are the receiver operating characteristic curves of each fold in K-fold cross-validation, and the dotted line is the receiver operating characteristic curve after the average of the aforementioned K receiver operating characteristic curves. The area under the curve is 0.9394.

A testing dataset of 87 cases of known healthy people (18 cases) and early-stage lung cancer group (69 cases) is collected. Healthy people refer to the subjects who have not been diagnosed with any major diseases, and the early-stage lung cancer group has not been treated. According to the analysis model for lung cancer early detection mentioned in the above examples, the cancer risk of each subject is determined, and the results listed in Table 3 below are obtained. Among the 87 test samples, the accuracy of successful determination of early lung cancer and healthy people is 94.25%, the sensitivity is 92.75%, and the specificity is 100%.

TABLE 3

| | | | | | | The area |
|---|---|---|---|---|---|---|
| | | | | | | under the |
| | | | | | | curve of |
| | | | | | | receiver |
| | | | | Positive | Negative | operating |
| Testing | | | | predictive | predictive | characteristic |
| dataset | Accuracy | Sensitivity | Specificity | value | value | curve |
| Isolated test | 0.9425 | 0.9275 | 1.0000 | 1.0000 | 0.7826 | 0.9887 |
| data | | | | | | |

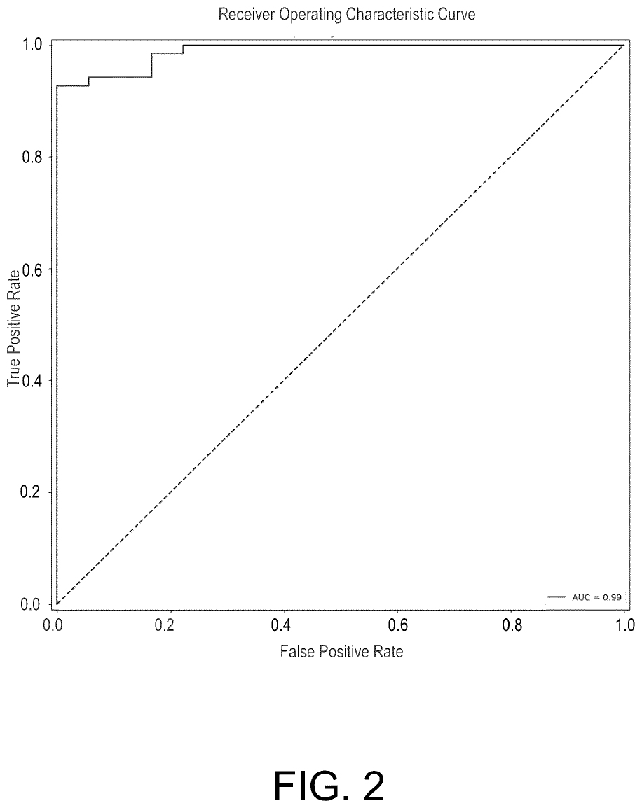

FIG. 2 is a receiver operating characteristic curve of an isolated test data (testing dataset). The black curve in FIG. 2 is the receiver operating characteristic curve of the isolated test data (testing dataset), and the area under the curve is 0.9887.

Based on the above, the invention provides a non-invasive cancer early detection method, in which the miRNA expression profile of a liquid biopsy sample of a subject is analyzed via the analysis model for cancer early detection. Therefore, early cancer screening may be performed in time and efficiently, and the convenience and detection rate of the conventional early cancer detecting technology may be improved, and personalized professional cancer detection and monitoring may be provided.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. A cancer early detection method, comprising:
establishing a miRNA expression profile database of cancer patient populations and healthy populations; and
establishing an analysis model for cancer early detection through following steps: a data quality control, a technical replicate merging, a calculation of normalization factors and data normalization, a biomarker feature selection, and a hyperparameter tuning, and the analysis model for cancer early detection includes normalization factors, a set of biomarkers, model weights and model hyperparameters;
wherein a miRNA expression profile in a liquid biopsy sample of a subject is analyzed by the analysis model for cancer early detection to be used as a basis for an early detection of cancer.

2. The cancer early detection method of claim 1, wherein the miRNA expression profile is determined by qPCR, sequencing, microarray, or RNA-DNA hybrid capture technology.

3. The cancer early detection method of claim 2, wherein the miRNA expression profile is determined by performing qPCR on a cDNA synthesized from a miRNA in the liquid biopsy sample.

4. The cancer early detection method of claim 1, wherein the miRNA expression profile comprises an expression level of a plurality of miRNAs.

5. The cancer early detection method of claim 1, wherein a type of the early detection of cancer comprises lung cancer.

6. The cancer early detection method of claim 1, wherein the liquid biopsy sample comprises plasma, serum, or urine, and exosomes further purified from the liquid biopsy sample.

7. The cancer early detection method of claim 1, wherein the analysis model for cancer early detection is established based on a classification algorithm, and the classification algorithm includes Logistic Regression or Random Forest.

8. The cancer early detection method of claim 1, wherein the data quality control comprises checking a ratio (a missing value ratio) of any miRNA in the miRNA expression profile database which does not have an expression level (a missing value) in all training dataset samples, and the miRNA of which the missing value ratio is 0 can be used as a normalization factor, if the normalization factor of a test sample contains the missing value, it is judged as failing the data quality control and removed from a testing dataset.

9. The cancer early detection method of claim 1, wherein when there are technical replicate data of a same sample in a training dataset or a testing dataset, the technical replicate merging is performed.

10. The cancer early detection method of claim 1, wherein the calculation of the normalization factor and the data normalization is to reduce a deviation between samples or batches by adjusting a data distribution or a normalization factor of a sample to be consistent.

11. The cancer early detection method of claim 1, wherein the biomarker feature selection is to remove noises caused by non-correlated features according to a weight or an importance of features.

12. The cancer early detection method of claim 1, wherein the hyperparameter tuning is used to find out a hyperparameter setting suitable for a data set.
